(19) United States
(12) Patent Application Publication
(10) Pub. No.: US 2020/0054397 A1
(43) Pub. Date: Feb. 20, 2020

(54) METHOD OF REDUCING STONE FRAGMENTS TO DUST DURING LASER LITHOTRIPSY

(71) Applicant: Optical Integrity, Inc., Panama City Beach, FL (US)

(72) Inventors: Joe D. Brown, Panama City Beach, FL (US); Daniel Malphurs, Panama City Beach, FL (US)

(73) Assignee: OPTICAL INTEGRITY, INC.

(21) Appl. No.: 16/546,992

(22) Filed: Aug. 21, 2019

Related U.S. Application Data
(63) Continuation-in-part of application No. 16/234,690, filed on Dec. 28, 2018.
(60) Provisional application No. 62/774,923, filed on Dec. 4, 2018.
(60) Provisional application No. 62/720,354, filed on Aug. 21, 2018.
(60) Provisional application No. 62/611,030, filed on Dec. 28, 2017.

(51) Int. Cl. A61B 18/26

(52) U.S. Cl. CPC A61B 18/26; A61B 17/22

(57) ABSTRACT

A soft tip is used to trap and pulverize stone fragments between the stone and the end of a single fiber, fiber bundle or lenticular array during a pulsed laser lithotripsy procedure.

This application claims the benefit of U.S. Provisional Patent Appl. Ser. No. 62/774,923, filed Dec. 4, 2018, and 62/720,354, filed Aug. 21, 2018, both of which are incorporated by reference herein.

This application is a continuation-in-part of copending U.S. patent application Ser. No. 16/234,690, filed Dec. 28, 2018, which claims the benefit of U.S. Provisional Patent Appl. Ser. No. 62/611,030, filed Dec. 28, 2017, and incorporated herein by reference.

### BACKGROUND OF THE INVENTION

### 1. Field of the Invention

The invention relates to the field of laser surgery, and in particular to a method of reducing a stone or other tissue to dust, also known as dusting, during a surgical laser procedure such as laser lithotripsy.

The method uses a spacer tip or standoff sleeve to maintain a predetermined distance from the stone. When the spacer tip or standoff is held in contact with the stone, a cavity is formed to trap and pulverize fragments of the stone as laser pulses pass through the cavity.

The spacer tip of standoff sleeve may be a compressible generally-cylindrical soft tip such as the one disclosed in copending PCT Appl. Ser. No. PCT/US2017/031091 (PCT Publ. No. WO/2017/192869), filed May 4, 2017, which is incorporated by reference herein. The spacer tip or standoff sleeve is fitted to an end of the optical fiber and extends beyond the distal end of the fiber to provide the predetermined spacing when the distal end of the soft tip is in contact with the stone,

The spacer tip may be maintained in contact with the stone by, for example, utilizing the methods disclosed in parent U.S. patent application Ser. No. 16/234,690, filed Dec. 28, 2018 (U.S. Pat. Publ. No. 2019/0201100), and U.S. patent application Ser. No. 16/353,225, filed Mar. 14, 2019, each of which is incorporated herein by reference.

The method may be applied to a single fiber, or to a fiber bundle or lenticular array to that direct multiple beams at the tissue or stone.

### 2. Description of Related Art

Laser lithotripsy is a surgical procedure to remove stones from urinary tract, i.e., kidney, ureter, bladder, or urethra, and was invented during the 1980s to remove impacted urinary stones. Early laser lithotripsy methods utilized pulsed-dye lasers with picosecond pulse durations to created cavitation bubbles that collapse and cause laser induced shockwaves with a high degree of retro-repulsion.

More recently, pulsed Holmium lasers have been developed with longer pulse durations (250 micro seconds) that produce a weaker pressure wave, and therefore less retro-repulsion, while still destroying the stones.

A problem with the use of pulsed lasers is that fragments of stone may break away, escape from the path of the laser during the interval between pulses, and therefore fail to be destroyed during the procedure. To address this problem, it has been proposed to use an array of optical fibers rather than a single fiber to deliver the laser energy to the stone or tissue to be destroyed. An example of a multiple fiber arrangement is disclosed in U.S. Pat. No. 5,395,361. Alternatively, it has been proposed to use a single fiber, and to separate the output beam into multiple beams by adding a lenticular array such as the one disclosed in U.S. Pat. No. 6,066,128, which is used in ophthalmic surgery.

Although the multiple beams provide a greater coverage area, however, it has been found that multiple beam arrangements can still leave fragments in a majority of procedures. A key determinant as to whether fragments are left appears to be the skill of the operator in maintaining an optimal spacing between the tip of the laser or the lenticular lens and the stone or tissue. Too large a spacing, whether due to operator error or retro-repulsion, can result in undesirable stone ablation. On the other hand, prolonged contact between the tissue or stone and the fibers or lenticular array can result in damage to the fibers or lenses.

The present can achieve greater pulverization efficiency than can be achieved by prior multiple beam arrangements, even with only a single pulsed laser beam, and can provide even greater dusting efficiency if multiple beams are used. The greater the efficiency by which stones and fragments are pulverized, the shorter the time required to carry out the procedure, resulting in decreased risk and less discomfort for the patient.

### SUMMARY OF THE INVENTION

The present invention is directed to a method of reducing a stone or other tissue to dust during a surgical laser procedure such as laser lithotripsy, both with and without the use of multiple fibers and lenticular arrays.

The method is to utilize a protective cap, and preferably a soft tip or spacer sleeve of the type described in PCT Publ. No. WO/2017/192869 to maintain a desired spacing between the end of the fiber and the stone while forming a cavity between the tip and the stone. The cavity traps fragments that have broken off the stone and therefore subjects them to repeated laser pulses that further reduce the size of the particles. The method may be applied to a single fiber, multiple fibers, or a fiber and lenticular array.

The spacer tip or standoff sleeve may optionally be maintained in contact with the stone by utilizing the methods disclosed in U.S. patent application Ser. Nos. 16/234,690, and 16/353,225, filed Mar. 14, 2019. As disclosed in U.S. patent application Ser. No. 16/234,690, for example, contact may be maintained by analyzing a spectrum to determine the stone-to-fiber distance based on the effect of vaporized material on the spectrum. However, it is also within the scope of the invention to maintain contact with the stone manually, based on operator observation during the lithotripsy procedure of stone position through the scope.

### BRIEF DESCRIPTION OF THE DRAWINGS

FIGS. 1A to 1C show the effect on stone fragments of a conventional pulsed laser.

FIGS. 2A to 2C show an embodiment of the invention that utilizes a soft tip to capture the fragments and subject them to multiple pulses.

FIGS. 3 and 4 show soft tips respectively fitted to a fiber bundle and lenticular array in accordance with the principles of preferred embodiments of the invention.

### DETAILED DESCRIPTION OF THE PREFERRED EMBODIMENTS

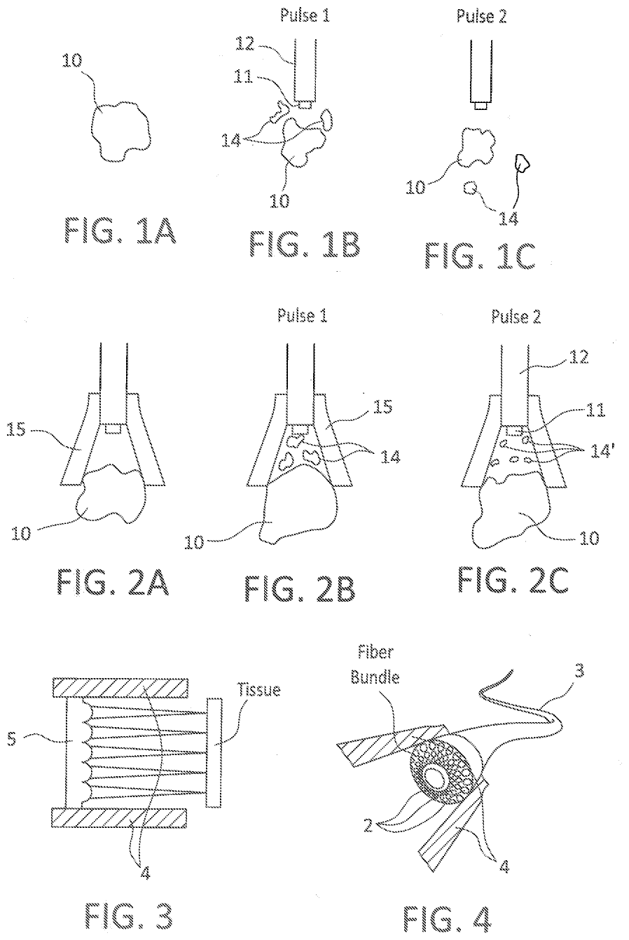

As shown in FIGS. 1A to 1C, a stone 10 in initially subjected to a first pulse emitted by the tip 11 of a fiber 12, resulting in the separation of fragments 14 from the stone 10. These fragments, like the stone is itself, are subject to retro-repulsion that, because of the lower mass of the fragments, tends to push them to the side or back behind the stone and out of reach of the laser. In order to dust these fragments, the operator must locate them and, in effect, chase them down by maneuvering the fiber tip into proximity to the fragments, even as the fragments continue to be pushed away from the fiber by retro-repulsion.

To solve this problem, a protective cap, spacer tip or standoff sleeve, preferably in the form of a compressible soft tip 15 of the type described in PCT Publ. No. WO/2017/192869, is secured to an end of the fiber such that the distal end of the protective cap extends beyond the end of the fiber, as illustrated in FIGS. 2A to 2C. As pulses are applied to the stone 10, the protective cap is maintained in contact with the stone, thereby trapping the fragments 14 that break off the stone so that the fragments are subjected to repeated pulses and reduced to smaller and smaller fragments 14′. Contact may be maintained manually by relying on operator observation of stone position and manual adjustment, or with the assistance of a stone position detection by spectral analysis as disclosed in

The protective cap, spacer tip or standoff sleeve may be manually maintained in contact with the stone, or optionally with the assistance of a proximity feedback method such as the one disclosed in copending U.S. patent application Ser. Nos. 16/234,690, and 16/353,225, cited above.

As illustrated in FIG. 2, the single fiber of FIG. 1A may be replaced by a fiber bundle 1 that includes a plurality of optical fibers 2 surrounded by a jacket 3. A distal end of the fiber bundle 1 is fitted with a cylindrical soft tip 4. During a lithotripsy procedure, the fiber bundle 1 Is inserted through a scope or catheter (not shown). As in the embodiment of FIG. 1A, when the end of the soft tip contacts the stone, the end of the fiber bundle 1 is spaced by a predetermined distance from the stone. The laser is fired while the soft tip is maintained in contact with the stone. Maintaining contact with the stone may again be accomplished by conventional methods, or with the assistance of a stone proximity-sensing method such as the described in copending U.S. patent application Ser. Nos. 16/234,690, and 16/353,225. The lithotripsy procedure continues until the stone and all fragments have been reduced to “dust,” i.e., fragments that are small enough to easily pass through the patient's urinary tract.

As illustrated in FIG. 2, fiber bundle 1 may be replaced by a single fiber or a smaller number of fibers, and at least one lenticular array 5 for splitting the laser output into a plurality of beams that achieve the same dusting effect as the plurality of beams emitted by the fiber bundle of FIG. 1. As in above-described embodiments, a soft tip 4 is fitted to the lenticular array 5 in order to provide a predetermined spacing between the array and the stone for optimal dusting effect.

What is claimed is:

1. A method of optimizing a dusting effect resulting from the firing of laser pulses from a laser deliver device at stone during a lithotripsy procedure, comprising the steps of:
placing a soft tip over a distal end of a laser delivery device, the soft tip extending between the distal end of the laser delivery device to provide a predetermined spacing between the stone and the distal end of the laser delivery device when the soft tip contacts the stone; and
firing laser beams at the stone until the stone has been reduced to dust while the soft tip is maintained in contact with the stone so as to trap any fragments that have broken off the stone in a space between a distal end of the fiber and the stone.

2. A method as claimed in claim 1, further comprising the step of using a stone sensing method to maintain contact between the soft tip and the stone during the lithotripsy procedure.

3. A method as claimed in claim 1, wherein the laser delivery device is a single optical fiber.

4. A method as claimed in claim 1, wherein the laser delivery device directs multiple beams at the stone and trapped fragments.

5. A method as claimed in claim 4, wherein the laser delivery device is a fiber bundle that includes a plurality of optical fibers.

6. A method as claimed in claim 4, wherein the laser delivery device includes a lenticular array positioned in front of at least one optical fiber for splitting a laser beam emitted by the at least one optical fiber into a plurality of laser beams.
